(19) United States
(12) Patent Application Publication
(10) Pub. No.: US 2023/0256081 A1
(43) Pub. Date: Aug. 17, 2023

(54) METHOD FOR PREPARING ATOMIZING SARS-COV-2 NANOVACCINE

(71) Applicants: Healthina Stem Cell Industry Platform (Tianjin) Limited, Tianjin (CN); Tangyi Holdings(Shenzhen) Limited, Shenzhen (CN)

(72) Inventors: Bin ZHENG, Tianjin (CN); Yulin CAO, Beijing (CN); Tingting HUA, Tianjin (CN); Shixiang CHENG, Tianjin (CN)

(73) Assignees: Healthina Stem Cell Industry Platform (Tianjin) Limited, Tianjin (CN); Tangyi Holdings(Shenzhen) Limited, Shenzhen (CN)

(21) Appl. No.: 17/720,334

(22) Filed: Apr. 14, 2022

(30) Foreign Application Priority Data
Feb. 17, 2022 (CN) 202210146014.9

(51) Int. Cl. A61K 39/215; A61P 31/14; A61K 9/00; A61K 9/51; A61K 39/39; A61K 9/127

(52) U.S. Cl. CPC A61K 39/215; A61P 31/14; A61K 9/0019; A61K 9/5123; A61K 39/39; A61K 9/1277; A61K 2039/55555

(57) ABSTRACT

A method for preparing an atomizing SARS-CoV-2 nanovaccine includes the followings steps: mimicking a structure of SARS-COV-2 with receptor binding domains (RBDs) of SARS-COV-2, by taking Poly(I:C) mimicking viral genetic materials as an immunoadjuvant, and electronegative liposomes that enter pulmonary macrophages efficiently as a viral capsid structure; adding a catalyst and an RBD antigen protein to a liposome solution, linking the antigen protein to a liposome surface, and obtaining a bionic virus nanovaccine after purification and freeze-drying treatment. Compared with conventional intramuscular and subcutaneous inoculation, the SARS-CoV-2 vaccine of the present invention features strong mucosal protection effect, high safety, extensive application, and excellent potential.

### CROSS REFERENCE TO THE RELATED APPLICATIONS

This application is based upon and claims priority to Chinese Patent Application No. 202210146014.9, filed on Feb. 17, 2022, the entire contents of which are incorporated herein by reference.

### TECHNICAL FIELD

The present invention relates to the technical field of nanovaccines mimicking virion, in particular to a method for preparing an atomizing SARS-CoV-2 nanovaccine.

### BACKGROUND ART

Novel coronavirus (SARS-CoV-2) features a strong transmission, a wide range of transmission and multiple infection routes. Vaccines are a powerful weapon in controlling the transmission of SARS-CoV-2. More than 100 SARS-CoV-2 vaccines are under development worldwide. At present, several vaccines available on the market are administrated intramuscularly. SARS-CoV-2 is mainly transmitted through the respiratory tract, while the existing immunization methods are difficult to induce respiratory mucosal immunity effectively. Meanwhile, the pain, complicated procedure and high cost of injection may lead to the problems of poor patient compliance, economic pressure, etc.

For patients in relatively underdeveloped countries, access to healthcare service is limited, the problems will be even more serious. The World Health Report listed the poor patient compliance and related medical waste issues such as needles and syringes as key issues in global immunization against deadly infectious diseases such as pneumococcal pneumonia. Besides, medical waste from multiple injections must be disposed of in a timely manner to avoid biohazard and the risk of disease transmission.

Similar to the influenza virus, SARS-CoV-2 mainly infects the human body through the respiratory tract. Therefore, respiratory mucosa is the first line of defense against respiratory virus invasion. As respiratory virus is different from the unique modes of infection and transmission of hepatitis virus, HIV virus, and rabies virus, stimulating respiratory mucosal immunity is one of the important ways to protect the body from viral infection. In view of these features, intranasal influenza vaccines emerging in recent years are expected to become a new therapeutic tool with great potential. After mucosal immunity is induced by intranasal vaccines, respiratory mucosal secretions contain a large amount of secretory immunoglobulins a, which can effectively neutralize the invading virus and make it unable to bind to receptor cells. Compared with nasal inhalation, intramuscular and subcutaneous injections are difficult to induce efficient mucosal immunity in subjects because antigens do not pass through the respiratory mucosa. Moreover, the nasal immunity can avoid pain and greatly reduce the risk of potential infection.

### SUMMARY

The present invention is intended to provide a method for preparing an atomizing SARS-CoV-2 nanovaccine, which provides a nanovaccine virion by mimicking a process of SARS-CoV-2 passing through the respiratory tract through inhalation, making full use of mucosal immunity serving as an important means to protect the body from respiratory virus infection. Compared with conventional intramuscular and subcutaneous inoculation, the SARS-CoV-2 vaccine of the present invention features strong mucosal protection effect, high safety, extensive application, and excellent potential in medicine.

To achieve the aforesaid purposes, the present invention provides a method for preparing an atomizing SARS-CoV-2 nanovaccine, including the following steps:

mimicking a structure of SARS-COV-2 with receptor binding domains (RBDs) of SARS-COV-2, by taking Poly(I:C) mimicking viral genetic materials as an immunoadjuvant, and electronegative liposomes that enter pulmonary macrophages efficiently as a viral capsid structure; and

adding a catalyst and an RBD antigen protein to a liposome solution, linking the antigen protein to a liposome surface, and obtaining a bionic virus nanovaccine after purification and freeze-drying treatment.

A method for preparing liposomes includes the following steps:

1) dissolving liposome components in an ethanol solution at a specific ratio as a solution A, and dissolving Poly(I:C) in a DNase/RNase-free water as a solution B; and

2) introducing the solution A and the solution B into a microfluidic emulsification instrument, and adjusting a velocity ratio of the solution A to the solution B to obtain liposomes with a uniform particle size.

A method for preparing a nanovaccine further includes the following steps:

1) dissolving the components (DPPG:DPPC:DPPE-PEG-COOH:cholesterol=1:10:1:1-3) of the liposome solution A in an ethanol solution, and the component of the solution B component is 0.5-2 mg/mL Poly(I:C) aqueous solution;

introducing the solution A and the solution B into two pipes of the microfluidic emulsification instrument respectively, and adjusting a velocity ratio of the solution A to the solution B to 4:1-1:1 to obtain liposome particles with an uniform size that enter pulmonary macrophages efficiently; and

2) adding EDC/NHS to the liposome solutions obtained in S1 for catalysis; then adding RBD proteins after reaction for 10-15 min, wherein a molar ratio of RBD:liposome is 1:1; and stirring the mixed solution at a room temperature for 0.5-2 h;

dialyzing the resulting mixed solution at 4° C. for 2-3 d to remove free RBD antigen proteins and catalyst to obtain a bionic virus nanovaccine solution, wherein the bionic virus nanovaccine obtained is preserved at −80° C. for a long time after being freeze-dried.

Therefore, the present invention adopts the above-mentioned method for preparing an atomizing SARS-CoV-2 nanovaccine, which has the following specific technical effects:

(1) The inhalable SARS-CoV-2 nanovaccine of the present invention contains capsid, nucleic acid and spike protein. The mucosal immunity is activated efficiently in a manner of nasal delivery, that is, the inhalable nanovaccine mimics a structure and invasion mode of SARS-CoV-2 as much as possible to prevent virus invasion, which can better induce the mucosal immunity of vaccinees.

(2) The nanovaccine mimicking virion of the present invention features a simple preparation method, convenient operation, modularity, and extensibility, and does not require high technical requirements. Moreover, compared with conventional intramuscular and subcutaneous inoculation, the nanovaccine features strong mucosal protection effect, high safety, and extensive application.

(3) The nanovaccine mimicking virion of the present invention can be inoculated by snuffing, which avoids a patient's large economic pressure, poor compliance and pain as a result of vaccine injection, and reduces the biohazard of needles/syringes and the risk of disease transmission.

The technical solutions of the present invention will be further described below in detail in combination with the accompanying drawings and embodiments.

### BRIEF DESCRIPTION OF THE DRAWINGS

FIG. 1 is a schematic diagram of a SARS-CoV-2 nanovaccine in the present invention.

FIG. 2 shows a charge stability of a SARS-CoV-2 nanovaccine in the present invention.

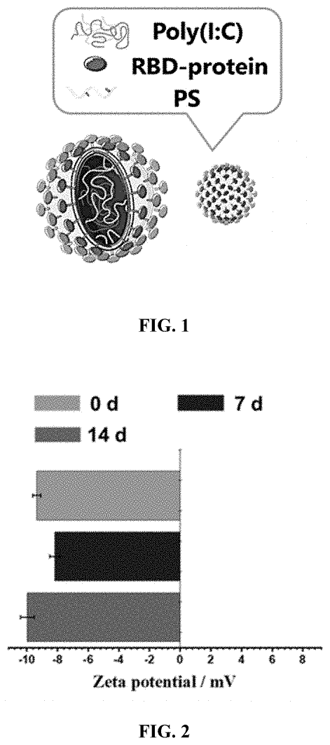

### DETAILED DESCRIPTION OF THE EMBODIMENTS

The technical solutions of the present invention will be further described below in combination with the accompanying drawings and embodiments.

Unless otherwise defined, the technical or scientific terms used herein should have ordinary meanings understood by those of ordinary skill in the art of the present invention.

It is apparent to those skilled in the art that the present invention is not limited to the details of the above-mentioned exemplary embodiments and can be realized in other specific forms without deviating from the intention or essential features of the present invention. Therefore, in all respects, the embodiments should be considered to be exemplary and non-restrictive. The scope of the present invention is limited by the appended claims rather than the above-mentioned description, so that all changes falling within the meaning and scope of the equivalents of the claims are intended to be included in the present invention, and any accompanying drawing marks in the claims should not be deemed to limit the claims involved.

Moreover, it should be understood that, although the specification is described according to the implementation modes, not each implementation mode contains only one independent technical solution. This narrative form of the specification is for the sake of clarity only. Those skilled in the art should take the specification as a whole, and the technical solutions in various embodiments may be combined appropriately to form other implementation modes that can be understood by those skilled in the art. These other implementation modes should also fall within the protection scope of the present invention.

In addition, it should be understood that the above-mentioned specific embodiments are used for explaining the present invention only, and the protection scope of the present invention is not limited to such specific embodiments. Within the technical scope disclosed by the present invention, the equivalent substitutions or changes made by those skilled in the art based on the technical solutions of the present invention and their inventive concept should fall within the protection scope of the present invention/invention.

The “including/comprising” or “containing” and similar words used herein mean that the element ahead of the word covers the element word and does not exclude the possibility of covering other elements as well. The orientations or position relations indicated by terms such as “inside”, “outside”, “up” and “down” indicate those shown based on the accompanying drawings, only used for the convenience of describing the present invention and simplifying the description, rather than indicating or implying that the device or element referred to must have, be constructed and operated in a particular orientation, so they cannot be understood as a limitation to the present invention. When the absolute position of the object described changes, the relative position relation may also change accordingly. In the present invention, unless otherwise expressly specified and limited, the term “attaching” should be understood in a broad sense. For example, two elements may be connected fixedly, connected detachably, or integrated; two elements may be connected directly, or connected indirectly through an intermediate medium, or communicated internally or interact. Those of ordinary skill in the art can understand the specific meanings of such terms in the present invention according to the specific situations. The term “about” used in the present invention has the meaning known to those skilled in the art, and preferably means that the value modified by the term is within the range of ±50%, ±40%, ±30%, ±20%, ±10%, ±5% or ±1%.

All terms (including technical or scientific terms) used in the disclosure have the same meanings as those understood by those of ordinary skill in the art of the disclosure, unless otherwise specifically defined. Moreover, it should be understood that terms defined in a general dictionary should be understood to have meanings consistent with those in the context of the relevant techniques, and should not be interpreted in an idealized or highly formal sense, unless expressly defined herein.

The techniques, methods and equipment known to those of ordinary skill in the art may not be discussed in detail, but where appropriate, such techniques, methods and equipment should be considered as a part of the specification.

The entire contents disclosed in the prior art literatures referenced in the specification of the present invention are incorporated herein by reference.

### EXAMPLES

I. Materials and Instruments

1,2-dipalmitoyl-sn-glycerol-3-phosphorylcholine (DPPC), (2,3-dioleoyloxy-propyl)-trimethylammonium-chloride; (2,3-dioleoyloxy-propyl)-trimethylammonium chloride, 1,2-dipalmitoyl-sn-glycero-3-phosphoethanolamine (DPPE), dipalmitoyl phosphatidyl glycerol (DPPG), cholesterol, (2,3-dioleoyloxy-propyl)-trimethylammonium-propane (DOTAP), and 1,2-dipalmitoyl-sn-glycerol-3-phosphorylethanolamine-n-[methoxy(polyethylene glycol)-1000]-COOH (DPPE-PEG1000-COOH, or DPPE-PEG-COOH): Purchased from Sigma-Aldrich; immunostimulant Poly(I:C): Purchased from MedChemExpress; TNF-α, IL-6 and IFN-0 (mouse) enzyme-linked immunosorbent assay reagents, and Commassie Blue Staining Kit: Purchased from AmyJet Scientific; SARS-CoV-2(2019-nCoV) spike pseudovirus, and RBD antigen: Purchased from Shanghai Genomic Technology Co., Ltd.; microfluidic emulsification instrument: Purchased from Suzhou Edson Pharmaceutical Equipment Co., Ltd.

### II. Preparation of Control Sample and Examples PS0-PS3

SARS-CoV-2 nanovaccines of the control sample and examples PS0-PS3 of the present invention were prepared according to the following general preparation process.

A general preparation method included the following steps:

liposomes carrying different charges including DPPG (negative charge), DOTAP (positive charge) and DPPE (neutral charge), together with DPPC, DPPE-PEG-COOH and cholesterol, were dissolved in ethanol respectively at a molar ratio of 2:10:1:1 as solution A, and Poly(I:C) mimicking viral genetic materials was dissolved in DNase/RNase-free water as solution B. The solution A and the solution B were introduced into a microfluidic emulsification instrument, and a velocity ratio of the solution A to the solution B was adjusted to 2:1 to obtain liposomes PS1/PS2/PS3.

EDC/NHS was added to the liposome solution for reaction for 15 min, and then RBD protein (RBD:liposome=1:1) was added, and the mixed solution was stirred at a room temperature for 1 h to obtain a bionic SARS-CoV-2 nanovaccine. PS0 group included free Poly(I:C) and RBD antigen of the same dose.

TABLE 1
Components of Control Sample and Examples PS0-PS3

| | Preparation of SARS-COV-2 Nanovaccines (Example) | | | |
|---|---|---|---|---|
| Group | PS0 | PS1 | PS2 | PS3 |
| Vaccine | Free | Negatively | Positively | Neutrally |
| components | Poly(I:C) + | charged | charged | charged |
| | RBD | liposome- | liposome- | liposome- |
| | | Poly(I:C)/ | Poly(I:C)/ | Poly(I:C)/RBD |
| | | RBD | RBD | |

TABLE 2
Characterization Data of PS0-PS3

| | Preparation of SARS-COV-2 Nanovaccines (Example) | | | |
|---|---|---|---|---|
| Group | PS0 | PS1 | PS2 | PS3 |
| Average | None | 108 nm | 105 nm | 112 nm |
| particle size | | | | |
| Average | 1 mV | −12 mV | 9 mV | 0 mV |
| potential | | | | |

### III. Immune Activation Example of SARS-CoV-2 Nanovaccines

SARS-CoV-2 nanovaccines were prepared from different vaccine components, and the efficiencies of such SARS-CoV-2 nanovaccines entering pulmonary macrophages and the immune activation ratios of such SARS-CoV-2 nanovaccines were tested. The results are as shown in Table 3.

TABLE 3
Immune Activation Ratios of SARS-COV-2 Nanovaccines
of Control Samples and Examples

| | SARS-COV-2 Nanovaccine | | | |
|---|---|---|---|---|
| Test | PS0 | PS1 | PS2 | PS3 |
| Endocytosis efficiency of | 10% | 86% | 75% | 53% |
| macrophages/% | | | | |
| Vaccine immune activation | 25% | 78% | 66% | 41% |
| ratio/% | | | | |

According to the data in Table 3, it can be seen that PS1 produced the largest endocytosis efficiency of macrophages and the highest vaccine immune activation ratio, and the vaccine components were negatively charged liposome-Poly(I:C)/RBD.

Therefore, the present invention adopts one of the above-mentioned methods for preparing an atomizing SARS-CoV-2 nanovaccine, which provides a nanovaccine mimicking virion by mimicking a process of SARS-CoV-2 passing through the respiratory tract through inhalation, making full use of mucosal immunity serving as an important means to protect the body from respiratory virus infection. Compared with conventional intramuscular and subcutaneous inoculation, the SARS-CoV-2 vaccine of the present invention features strong mucosal protection effect, high safety, extensive application, and excellent potential in medicine.

Finally, it should be stated that the above-mentioned embodiments are only used for describing, rather than limiting, the technical solutions of the present invention. Although the present invention is described in detail by reference to the preferred embodiments, those of ordinary skill in the art should understand that they can still make modifications or equivalent substitutions to the technical solutions of the present invention, but these modifications or equivalent substitutions will not make the modified technical solutions deviate from the spirit and scope of the technical solutions of the present invention.

What is claimed is:

1. A method for preparing an atomizing SARS-CoV-2 nanovaccine, comprising the following steps:
1) mimicking a structure of SARS-COV-2 with receptor binding domain (RBD) antigen proteins of the SARS-COV-2, by taking Poly(I:C) mimicking viral genetic materials as an immunoadjuvant, and electronegative liposomes as a viral capsid structure, wherein the electronegative liposomes are configured for entering pulmonary macrophages efficiently; and
2). adding a catalyst and the RBD antigen proteins to a solution of the electronegative liposomes, linking the RBD antigen proteins to a liposome surface, and performing a purification and freeze-drying treatment to obtain a bionic virus nanovaccine.

2. The method for preparing the atomizing SARS-CoV-2 nanovaccine according to claim 1, wherein the electronegative liposomes in step 1 are prepared by a method comprising the following steps:
1.1) dissolving liposome components in an ethanol solution at a specific ratio to obtain a first solution, and dissolving the Poly (I:C) mimicking viral genetic materials in DNase/RNase-free water to obtain a second solution; and
1.2) introducing the first solution and the second solution into a microfluidic emulsification instrument, and adjusting a velocity ratio of the first solution to the second solution to obtain the electronegative liposomes with a uniform particle size.

3. The method for preparing the atomizing SARS-CoV-2 nanovaccine according to claim 2, comprising the following steps:
1.1) dissolving the liposome components of the first solution in the ethanol solution, and dissolving the Poly (I:C) mimicking viral genetic materials in the DNase/RNase-free water to obtain the second solution, wherein the liposome components comprise dipalmitoyl phosphatidyl glycerol (DPPG), 1,2-dipalmitoyl-sn-glycerol-3-phosphorylcholine (DPPC), 1,2-dipalmitoyl-sn-glycerol-3-phosphorylethanolamine-n-[methoxy (poly ethylene glycol)-1000]-COOH (DPPE-PEG-COOH), and cholesterol, wherein a molar ratio of the DPPG: the DPPC: the DPPE-PEG-COOH: the cholesterol is 1:10:1: (1-3), and the second solution is a 0.5-2 mg/mL Poly(I:C) aqueous solution;
1.2) introducing the first solution and the second solution into two pipes of the microfluidic emulsification instrument, respectively, and adjusting the velocity ratio of the first solution to the second solution to 4:1-1:1 to obtain the electronegative liposomes with the uniform particle size; and
2) adding EDC/NHS to the solution of the electronegative liposomes obtained in step 2 for a catalysis reaction, then after 10-15 mins, adding the RBD antigen proteins, and stirring at a room temperature for 0.5-2 h to obtain a mixed solution; and
dialyzing the mixed solution at 4° C. to remove free RBD antigen proteins and free catalyst to obtain a solution of the bionic virus nanovaccine.

4. The method for preparing the atomizing SARS-CoV-2 nanovaccine according to claim 3, wherein a molar ratio of the RBD antigen proteins to the electronegative liposomes in step 2 is 1:1.

5. The method for preparing the atomizing SARS-CoV-2 nanovaccine according to claim 3, wherein a dialysis time in step 2 is 2-3 days.

6. The method for preparing the atomizing SARS-CoV-2 nanovaccine according to claim 3, wherein the bionic virus nanovaccine obtained in step 2 is freeze-dried and preserved at −80° C.
